(19) United States
(12) Patent Application Publication
(10) Pub. No.: US 2017/0014375 A1
(43) Pub. Date: Jan. 19, 2017

(54) PHARMACEUTICAL COMPOSITION COMPRISING EFFECTIVE DOSE OF POMIFERIN FOR TREATING CANCERS

(71) Applicant: Macau University of Science and Technology, Macau (MO)

(72) Inventors: Kam Wai WONG, MO (MO); Yuen Kwan LAW, MO (MO); Liang LIU, MO (MO); Su-Wei XU, MO (MO)

(21) Appl. No.: 14/799,541

(22) Filed: Jul. 14, 2015

(51) Int. Cl. A61K 31/352

(52) U.S. Cl. CPC A61K 31/352

(57) ABSTRACT

The present invention provides a compound of formula (I) as a SERCA inhibitor for treating cancers, a pharmaceutical composition comprising said compound, and methods of using said compound for treating cancers and/or inducing cell death in cells of said cancers. Said cancers include but not limited to cervical, lung, liver, breast, and prostate cancer. Said cancers also include drug-resistant and/or apoptosis-resistant cancers such as isogenic drug-resistant colon cancer. The subject being administered with said compound or the composition comprising thereof can be human or animal subject. Said methods for treating cancers and/or inducing cell death can be a targeting treatment for certain cancers.

### FIELD OF INVENTION

The present invention relates to a method of using a compound for treating cancers comprising applying an effective amount of said compound to a subject for inducing cell death in drug-resistant and/or apoptosis-resistant cancer. In particular, the present invention relates to a method of using said compound as SERCA inhibitor for treating cancers comprising applying an effective amount of said compound for inhibiting SERCA via calcium mobilization and autophagy induction, thereby inducing cell death in drug-resistant and/or apoptosis-resistant cancer. The present invention also provides a pharmaceutical composition comprising an effective amount of said compound for treatment of cancers including drug-resistant and/or apoptosis-resistant cancer.

### BACKGROUND OF INVENTION

Therapeutic target proteins with heterogeneous expression pattern in cancer cells usually lead to drug resistance phenotype, which becomes the major obstacle in the treatment of cancer via target therapy¹. Clinical therapies handling this heterogeneous issue are limited and therefore, small-molecules that retain effectiveness against drug-resistant cancers are urgently demanded. Specific inhibition of the sarcoplasmic/endoplasmic reticulum Ca²⁺ ATPase (SERCA) calcium pump would be a promising approach to circumvent this problem because the continued expression and calcium transport function of SERCA are crucial to the survival of all cancer cells². Previous studies indicated that suppression of SERCA could mobilize cytoplasmic calcium in cancer cells, thereby induces endoplasmic reticulum (ER)-stress response and contributes permanent mitochondrial damage by Ca²⁺ overload, leading to cell death induction¹. Most importantly, SERCA inhibitors exhibit potent anti-cancer effect toward Bax- and Bak-deficient apoptosis-resistant tumors and other multi-drug resistance (MDR) cancer cells³⁻⁵, which further highlight the remarkable application of SERCA inhibitors as promising anti-cancer agents for the treatment of drug-resistant tumors.

Autophagy is a cellular degradation process that involves the delivery of cytoplasmic cargo such as long-lived protein, mis-folded protein or damaged organelles, sequestered inside double-membrane vesicles to the lysosome. Autophagy occurs at low basal levels in cells to maintain normal homeostatic functions by protein and organelle turnover. Upon cellular stressful conditions such as nutrient deprivation, oxidative stress, infection or protein aggregate accumulation, autophagy starts with membrane isolation and expansion to form the double-membraned vesicle (autophagosome) that sequesters the cytoplasmic materials. Followed by fusion of the autophagosome with lysosome to form an autolysosome, all the engulfed materials are degraded to recycle intracellular nutrients and energy⁶. Impaired autophagy and the age-related decline of this pathway favour the pathogenesis of many diseases that occur especially at higher age such as cancers and neurodegenerative diseases⁷. While autophagy may play a protective role in neurodegenerative disease⁸, autophagic dysfunction is associated with DNA damage, chromosome instability^{9,10}, and increased incidence of malignancies¹⁰. Modulators of autophagy may play a protective role through promoting autophagic cell death in tumors or augment the efficacy of chemotherapeutic agents when used in combination. Several clinically approved or experimental antitumor agents induced autophagy-related cell death^{2,11-13}. Indeed, inhibition of SERCA could activate calcium-mediated autophagy induction in cancer cells, thereby induces autophagic cell death in cancer cells and apoptosis-resistant cells². Therefore, there is a need for cancer treatment strategies that can overcome apoptosis resistance and multi-drug resistance in cancers.

Pomiferin is a unique, prenylated isoflavonoid that can be isolated and purified from the fruits of Maclura pomifera (Osage Orange)¹⁴. Studies reported that it exhibits various biological activities such as anti-oxidant¹⁵, anti-fungal¹⁶, anti-cancer^{17,18} and anti-diabetic¹⁹. However, the mechanistic action on anti-cancer and the molecular target of pomiferin are unclear.

### SUMMARY OF INVENTION

Accordingly, in the present invention, a method of using a compound having the following formula:

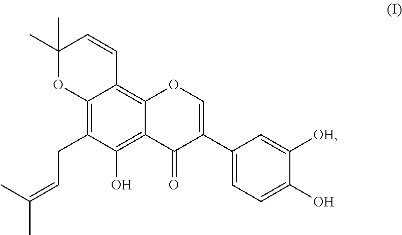 [SMILES: CC(C)=CCc1c2c(c3occ(-c4ccc(O)c(O)c4)c(=O)c3c1O)C=CC(C)(C)O2]

which is also named as pomiferin, in anti-cancer treatment, especially effective on apoptosis-resistant and drug-resistant cancers, is provided.

It is an objective of the present invention to provide said compound of formula (I) as a SERCA inhibitor for use in inducing cell death in cancers including drug-resistant and/or apoptosis-resistant cancers. In particular, it is an objective of the present invention to provide a pharmaceutical composition comprising an effective amount of said compound of formula (I) as a SERCA inhibitor for use in inducing cell death in cancer cells, leading to treatment for the cancers. Said use or method of treating cancers including drug-resistant and/or apoptosis-resistant cancers comprises administering an effective amount of said compound to a subject in needs thereof, said subject can be an animal or human. In one embodiment, the effective amount of said compounds ranges from 3.82 to 20.3 μM and the composition is administered for at least 72 hours; said cancers include cancer cells from cancers of human or animal subject. In another embodiment, the effective amount of said compound ranges from 0.44 to 7.63 μM and said cancers include apoptosis-resistant cancer cells from human or mouse origin. In other embodiment, the effective amount of said compound ranges from 5.58 to 6.51 μM and said cancers include isogenic drug-resistant cancer cells from human or mouse origin. In yet another embodiment, the pharmaceutical composition further comprises a pharmaceutically acceptable carrier, salt, buffer, water, or a combination thereof.

The present invention is first to demonstrate the SERCA inhibition activity, calcium mobilization and autophagic effect of said compound. The present invention also demonstrates potent cytotoxic activity of said compound towards a panel of cancer cells. In particular, the cancer cells are human or mouse cancer cells. The compound of formula (I) can be chemically synthesized or isolated from the fruits of Maclura pomifera.

In a first aspect, the present invention relates to a method of inducing cell death in cancer cells, said method comprising exposing the cells to an effective amount of pomiferin that mobilizes cytosolic calcium and induces autophagy in said cells. Said induction of cell death includes an apoptosis and autophagic cell death. Said cells comprise apoptosis-resistant cells, drug-resistant cells or cancer cells. In one embodiment, said apoptosis-resistant cells, drug-resistant cells or cancer cells are originated from human or mouse. In another embodiment, the present method of inducing cell death further comprises selectively targeting apoptosis-resistant cancer cells, drug-resistant cancer cells or cancer cells only.

In a second aspect, the present invention provides a composition for use in the treatment of cancer comprising an effective amount of the compound of Formula (I), wherein said composition is administered to a subject in needs thereof to inhibit SERCA activity in cancer cells of said cancer. Inhibition of SERCA activity leads calcium mobilization and autophagy related cell death in said cancer. Said subject includes human subject and said human subject either has drug resistance to conventional therapeutic agents which induce cell death in cancer cells, or tends to have said drug resistance. Said cells include apoptosis-resistant cancer cells, drug-resistant cancer cells or cancer cells. Said compound or composition may only target said apoptosis-resistant cancer cells, drug-resistant cancer cells or cancer cells in said subject in order to provide a cancer-specific treatment.

### BRIEF DESCRIPTION OF FIGURES

The patent or application file contains at least one drawing executed in color. Copies of this patent or patent application publication with color drawing(s) will be provided by Office upon request and payment of the necessary fee.

FIGS. 1A & B: Pomiferin induces autophagic LC3-II conversion via autophagic flux: FIG. 1A shows LC3-II expression level under different concentrations (0, 5, 10, and 15 μM) of pomiferin in HeLa cells; FIG. 1B shows difference in LC3-II expression between 10 μM pomiferin and 50 nM bafilomycin A, a lysosomal inhibitor, in HeLa cells.

FIG. 1C shows that autophagy inhibitor, 3-methyladenine (3-MA), abolishes pomiferin-mediated autophagic LC3-II conversion: upper panel: western blot result shows that expression of LC3-II is inhibited by 5 mM 3-MA even in the presence of 10 μM pomiferin; lower panel: a graph shows the fold change in LC3-II expression in different groups of treatment.

FIG. 1D shows that autophagy inhibitor, 3-MA, inhibits pomiferin-mediated autophagic GFP-LC3 puncta formation in HeLa cancer cells: upper left, upper right and lower left panels: immunofluorescent images of HeLa cells with GFP-LC3 expression in control, 10 μM pomiferin, and 5 mM 3-MA; lower right panel: % of cells with GFP-LC3 expression.

FIG. 2 is a series of fluorescent images for TRITC-LC3 puncta formation in different normal and cancer cells treated with 10 μM pomiferin revealing that pomiferin induces endogenous autophagic effect in normal or cancer cells.

FIG. 3A is a result of western blot showing down-regulation of p70S6K and up-regulation of AMPK, revealing that pomiferin activates AMPK-mTOR signaling pathways.

FIG. 3B is a result of western blot for LC3-II expression in HeLa cells treated with 5 μM AMPK inhibitor, compound C, and/or 10 μM pomiferin, revealing that compound C abrogates pomiferin-mediated autophagic LC3-II conversion.

FIG. 3C is a series of fluorescent images and a graph for GFP-LC3 expression in HeLa cells treated with 5 μM compound C and/or 10 μM pomiferin revealing that compound C suppresses pomiferin-mediated autophagic GFP-LC3 puncta formation in HeLa cancer cells.

FIG. 3D is a western blot result for LC3-II expression in HeLa cells treated with 25 μM CaMKK-β inhibitor, STO-609, and/or 10 μM pomiferin, revealing that STO-609 abrogates pomiferin-mediated autophagic LC3-II conversion.

FIG. 3E is a series of fluorescent images and a graph for GFP-LC3 expression in HeLa cells treated with 25 μM STO-609 and/or 10 μM pomiferin, revealing that STO-609 suppresses pomiferin-mediated autophagic GFP-LC3 puncta formation in HeLa cancer cells.

FIG. 4A is a series of histograms of a flow cytometry study and a graph on cytosolic calcium release from HeLa cells at different time points (10 min, 0.5 hr, 1 hr, 2 hr, and 4 hr) over a time course treated with 10 μM pomiferin, showing that pomiferin mobilizes cytosolic calcium level in HeLa cancer cells.

FIG. 4B is a western blot result and a graph for LC3-II expression in HeLa cells treated with 10 μM calcium chelator, BAPTA/AM, and/or 10 μM pomiferin, revealing that BAPTA/AM abolishes pomiferin-mediated autophagic LC3-II conversion.

FIG. 4C is a series of fluorescent images and a graph for GFP-LC3 expression in HeLa cells treated with 10 μM BAPTA/AM and/or 10 μM pomiferin, revealing that BAPTA/AM suppresses pomiferin-mediated autophagic GFP-LC3 puncta formation in HeLa cancer cells.

FIG. 5 is a 3-D computational docking predicting binding site or target of pomiferin on SERCA: FIG. 5A shows that thapsigargin (TG) is the positive control drug to show the drug binding site on SERCA in this example; FIG. 5B is a curve showing the result of a SERCA activity assay against different concentrations of pomiferin.

FIG. 6A are fluorescent images and a graph showing that pomiferin-induced autophagy is dependent on the presence of autophagy-related gene 7 (Atg7); fluorescent signal of endogenous LC3-II puncta is detected in mouse embryonic fibroblasts (MEF) cells with wild-type Atg7 while no signal is detected in autophagy-deficient (Atg7^{-/-}) MEF cells which are both treated with 2 μM pomiferin.

FIG. 6B is a series of two-parameter histograms of a flow cytometry analysis and two graphs for expression of Annexin V and percentage of cell death in Atg7^{+/+} and Atg7^{-/-} MEF cells treated with 2 μM pomiferin; both flow cytometry result and expression level in two graphs, revealing that pomiferin-mediated cell death is dependent on autophagy induction.

FIG. 7 is a series of fluorescent images showing that pomiferin induces endogenous autophagic effect in apoptosis-resistant cells; red fluorescent signal represents LC3-II conversion under the treatment of pomiferin in apoptosis-resistant cells including caspase wild-type (caspase WT), caspase-3 deficient (caspase 3KO), caspase-7 deficient (caspase 7KO), caspase-3/-7 deficient (caspase 3/7 DKO), caspase-8 deficient (caspase 8KO), Bax-Bak wild-type (Bax-Bak WT) and Bax-Bak double knock out (Bax-Bak DKO) MEFs.

FIG. 8A is a series of flow cytometry histograms and two graphs for expression of annexin V and percentage of cell death in Bax-Bak WT and Bax-Bak DKO MEFs treated with 5 μM pomiferin, revealing that pomiferin exhibits collateral sensitivity in Bax-Bak DKO apoptosis-resistant cells.

FIG. 8B is a series of flow cytometry histograms and two graphs for expression of annexin V and percentage of cell death in Bax-Bak WT and Bax-Bak DKO MEFs treated with 5 μM pomiferin and/or 5 mM 3-MA, revealing that 3-MA markedly abrogates pomiferin-mediated cell death in apoptosis-resistant cells.

FIG. 9A is a series of flow cytometry histograms and two graphs for expression of annexin V and percentage of cell death in HCT116 p53−/− cells treated with 10 μM pomiferin and/or 5 mM 3-MA, revealing that 3-MA markedly abrogates pomiferin-mediated cell death in drug-resistant cancer, HCT-116 p53^{-/-}.

FIG. 9B is a series of flow cytometry histograms and a graph for expression of annexin V and percentage of cell death in HCT116 p53−/− cells treated with 10 μM pomiferin and/or 10 μM BAPTA/AM, revealing that BAPTA/AM significantly suppresses pomiferin-mediated cell death in drug-resistant cancer cells, HCT-116 p53−/−.

### DETAILED DESCRIPTION OF THE PREFERRED EMBODIMENTS

The following preparations and examples are given to enable those skilled in the art to more clearly understand and to practice the present invention. They should not be considered as limiting the scope of the invention, but merely as being illustrative and representative thereof.

### Example 1

### In Vitro Cytotoxicity Test of Pomiferin in a Panel of Human or Mouse Cancer Cells

Cell Culture and Cytotoxicity Assay:

Pomiferin is dissolved in DMSO at a final concentration of 100 mmol/L and stored at −20° C. Cytotoxicity is assessed using the 3-(4,5-dimethylthiazol-2-yl)-2,5-diphenyltetrazolium bromide assay. 4000-8000 HeLa (human cervical cancer), MCF-7 (human breast cancer), HepG2 and Hep3B (human liver cancer), H1299 and A549 (human lung cancer), LNCap (human prostate cancer) and LLC-1 (mouse Lewis lung carcinoma) cells are seeded on 96-well plates per well. After overnight pre-incubation, the cells are exposed to different concentrations of pomiferin (0.039-100 mol/L) for 3 days. Subsequently, 10 μL of MTT reagents is added to each well and incubated at 37° C. for 4 hours followed by the addition of 100 μL solubilization buffer (10% SDS in 0.01 mol/L HCl) and overnight incubation. Absorbance at 585 nm is determined from each well the next day. The percentage of cell viability is calculated using the following formula: Cell viability (%)=Cells number treated/Cells number DMSO control×100. Data are obtained from three independent experiments.

Results:

There is significant cell cytotoxicity with mean IC₅₀ value ranging from 3.82-20.3 μM observed in a panel of human and mouse cancer cells treated with pomiferin for 72 hours, which is revealed by MTT assay (Table 1).

TABLE 1
Cell cytotoxicity of pomiferin towards a panel
of cancer cells in terms of mean IC₅₀ value:

| Cell line | Means of IC₅₀ [μM] |
|---|---|
| HeLa (Cervical) | 7.36 |
| MCF-7 (Breast) | 19 |
| HepG2 (Liver) | 13.6 |
| Hep 3B (Liver) | 16.3 |
| A549 (Lung) | 20.3 |
| LNcap (Prostate) | 16.2 |
| H1299 (Lung) | 16.3 |
| LLC-1 (Lung) | 3.82 |

### Example 2

### Pomiferin Induces Autophagic Flux and Endogenous Autophagic Puncta in Cancer Cells

Detection of Autophagic Flux by Pomiferin:

After pomiferin treatments in the presence or absence of lysosomal inhibitor 50 nM of Bafilomycin A1 or autophagy inhibitor 5 mM 3-MA, HeLa cancer cells are harvested and lysed in RIPA buffer (Cell Signaling Technologies Inc., Beverly, Mass.). The cell lysates are then resolved by SDS-PAGE. After electrophoresis, the proteins from SDS-PAGE are transferred to nitrocellulose membrane which is then blocked with 5% non-fat dried milk for 60 minutes. The membrane is then incubated with LC3 primary antibodies (1:1000) in TBST overnight at 4° C. After that, the membrane is further incubated with HRP-conjugated secondary antibodies for 60 minutes. Finally, protein bands are visualized by using the ECL Western Blotting Detection Reagents (Invitrogen, Paisley, Scotland, UK).

Quantification of Autophagy GFP-LC3 Puncta:

GFP-LC3 puncta formation is quantified as previously described¹³. In brief, cells grown on coverslips in a 6-well plate are treated with or without 10 μM of pomiferin for 24 hours, the cells are then fixed in 4% paraformaldehyde for 20 minutes at room temperature and then rinsed with PBS. Slides are mounted with FluorSave™ mounting media (Calbiochem, San Diego, Calif.) and examined by fluorescence microscopy. The number of GFP-positive cells with GFP-LC3 puncta formation is captured and examined under the Delta Vision fluorescence microscope. To quantify for autophagy, the percentage of cells with punctate GFP-LC3 fluorescence is calculated by counting the number of the cells with punctate GFP-LC3 fluorescence in GFP-positive cells. A minimum of 150 cells from 3 randomly selected fields is scored.

Results:

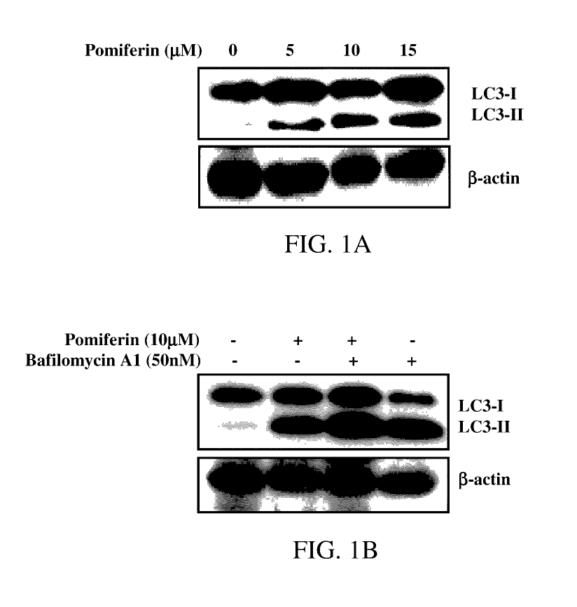

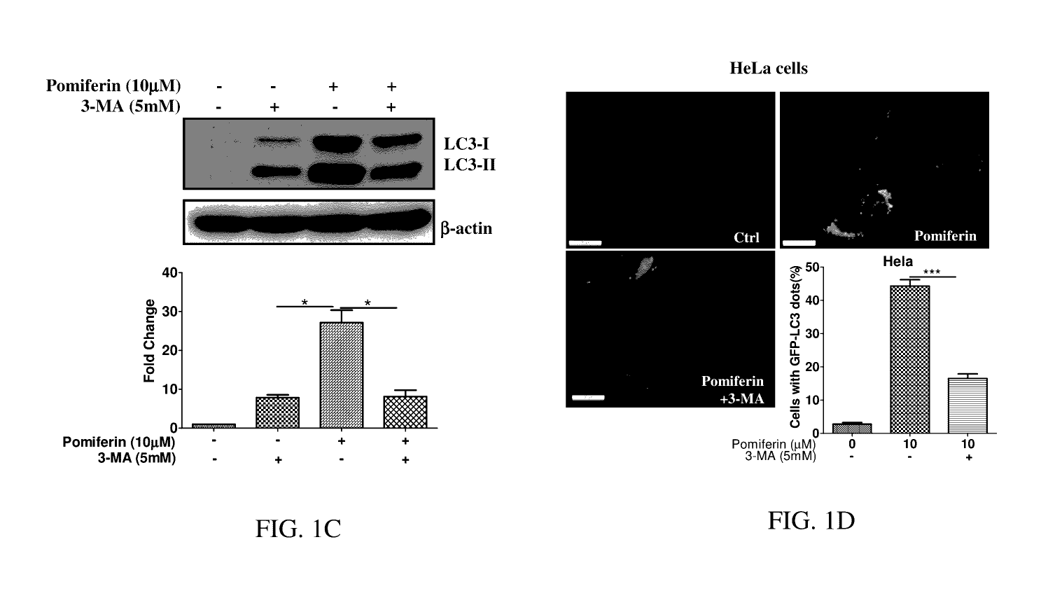

Western blot analysis showed that the autophagic marker LC3-II conversion is induced upon pomiferin treatment as shown in FIG. 1A. In addition, pomiferin is able to further enhance the LC3-II conversion in the presence of lysosomal inhibitor (Bafilomycin A1) as illustrated in FIG. 1B. Collectively, these data suggest that pomiferin is able to induce autophagy via increasing of autophagic flux. On the other hand, autophagic inhibitor, 3-MA is used to confirm and validate the pomiferin-mediated autophagy. Obviously, addition of 3-MA could markedly suppress the pomiferin-mediated LC3-II conversion. Bar chart represented the quantitation of LC3-II protein conversion (FIG. 1C). Besides, FIG. 1D further demonstrated that the pomiferin-mediated GFP-LC3 autophagic puncta formation is significantly inhibited by 3-MA. Collectively, pomiferin is confirmed to induce autophagy in cancer cells via autophagic flux.

### Example 3

### Pomiferin Induces Endogenous LC3 Puncta Formation in a Panel of Cancer and Normal Cells

Endogenous Autophagy Detection:

The detection of endogenous LC3 puncta formation is conducted using immunofluorescence staining method as described below. In brief, pomiferin-treated cancer cells (Hep3B, HepG2, H1299, MCF-7 and A549) or normal human hepatocyte (LO2) on cover slips are fixed with 4% paraformaldehyde (Sigma) for 20 min at room temperature and then rinsed with PBS. Immerse coverslips in methanol at room temperature for 2 min. After ishing with PBS, the cells are then incubated with anti-LC3 (1:200) in TBST (100 mM Tris HCl, pH 7.5, 150 mM NaCl, 0.05% Tween 20 and 5% BSA) overnight at 4. After washing with PBS, the cells are incubated with anti-mouse secondary antibody (TRITC) 1:200 in TBST containing 5% BSA at 37 for 1 hrs in the dark. The coverslips are then mounted with FluorSave™ mounting media (Calbiochem, San Diego, Calif., USA) for fluorescence imaging and localization of LC3 autophagosomes are captured under the API Delta Vision Live-cell Imaging System (Applied Precision Inc., GE Healthcare Company, Washington, USA). To quantify autophagy, guidelines are followed to monitor autophagy²⁰, the percentage of cells with punctuate LC3 immunofluorescence staining is calculated by counting the number of the cells showing the punctuate pattern of LC3 fluorescence (>10 dots/cell) in immunofluorescence positive cells over the total number of cells in the same field. A minimum of 1000 cells from randomly selected fields are scored.

Results:

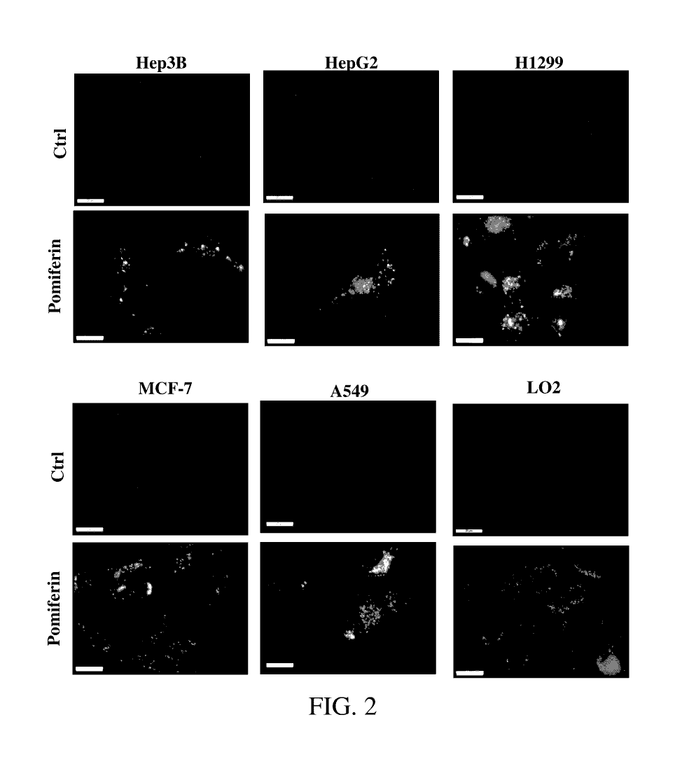

FIG. 2 indicated that pomiferin induces TRITC-LC3 puncta formation in all tested cancer and normal cells, suggesting that the pomiferin-induced autophagy is not cell-type specific.

### Example 4

### Pomiferin Induces Autophagy Via Activation of AMPK-mTOR Signaling Cascade

Detection of mTOR Signaling Marker Proteins:

HeLa cancer cells treated with indicated time and concentrations of pomiferin are harvested and lysed in RIPA buffer (Cell Signaling). The cell lysates are then resolved by SDS-PAGE. After electrophoresis, the proteins from SDS-PAGE are transferred to nitrocellulose membrane which is then blocked with 5% non-fat dried milk for 60 minutes. The membrane is then incubated with P-p70S6K, p70S6K, P-AMPK, AMPK and actin primary antibodies (1:1000) in TBST overnight at 4° C. respectively. After that, the membrane is further incubated with HRP-conjugated secondary antibodies for 60 minutes. Finally, protein bands are visualized by using the ECL Western Blotting Detection Reagents (Invitrogen).

Detection of Autophagic Marker Protein LC3-II Conversion:

After pomiferin treatment in the presence or absence of AMPK inhibitor compound c [5 μM] or CaMKK-β inhibitor STO-609 [25 μM], HeLa cancer cells are harvested and lysed in RIPA buffer (Cell Signaling Technologies Inc. (Beverly, Mass.). The cell lysates are then resolved by SDS-PAGE. After electrophoresis, the proteins from SDS-PAGE are transferred to nitrocellulose membrane which is then blocked with 5% non-fat dried milk for 60 minutes. The membrane is then incubated with LC3 primary antibodies (1:1000) in TBST overnight at 4° C. After that, the membrane is further incubated with HRP-conjugated secondary antibodies for 60 minutes. Finally, protein bands are visualized by using the ECL Western Blotting Detection Reagents (Invitrogen, Paisley, Scotland, UK).

Quantification of Autophagy GFP-LC3 Puncta:

GFP-LC3 puncta formation is quantified as previously described¹³. In brief, cells grown on coverslips in a 6-well plate are incubated with 10 μM of pomiferin in the presence or absence of AMPK inhibitor compound c [5 μM] or CaMKK-β inhibitor STO-609 [25 μM] for 24 hours, the cells are then fixed in 4% paraformaldehyde for 20 minutes at room temperature and then rinsed with PBS. Slides are mounted with FluorSave™ mounting media (Calbiochem, San Diego, Calif.) and examined by fluorescence microscopy. The number of GFP-positive cells with GFP-LC3 puncta formation is captured and examined under the Delta Vision fluorescence microscope. To quantify for autophagy, the percentage of cells with punctate GFP-LC3 fluorescence is calculated by counting the number of the cells with punctate GFP-LC3 fluorescence in GFP-positive cells. A minimum of 150 cells from 3 randomly selected fields is scored.

Results:

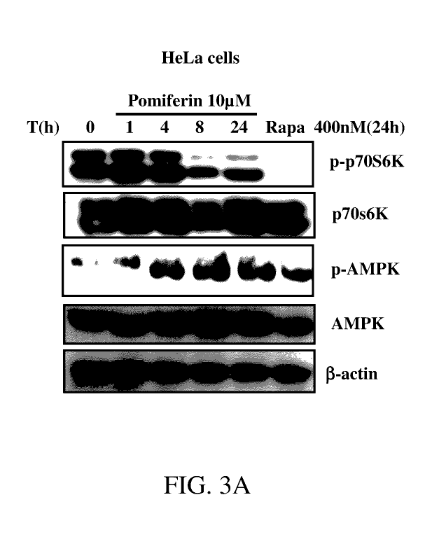

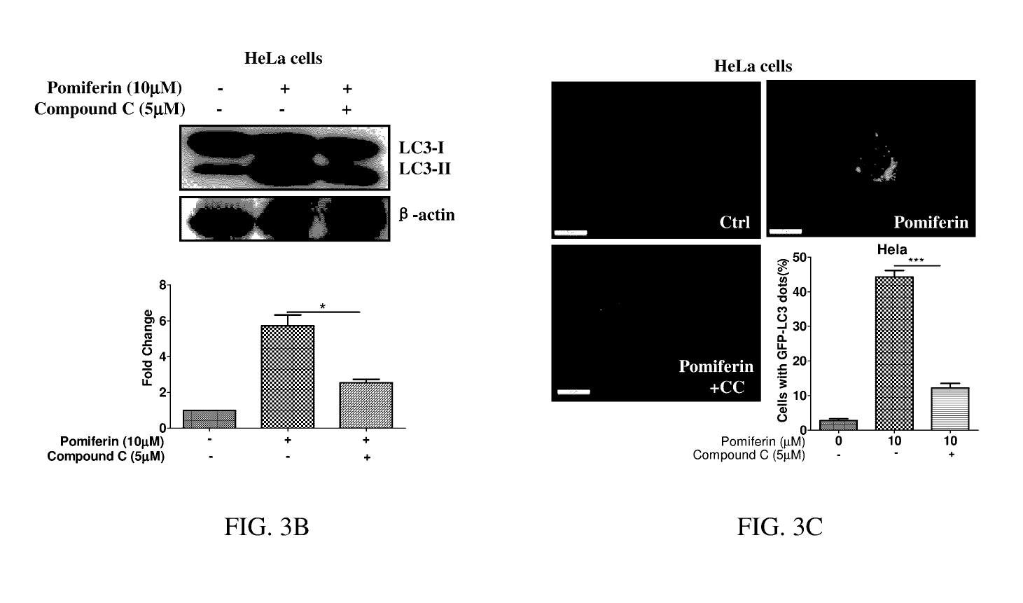

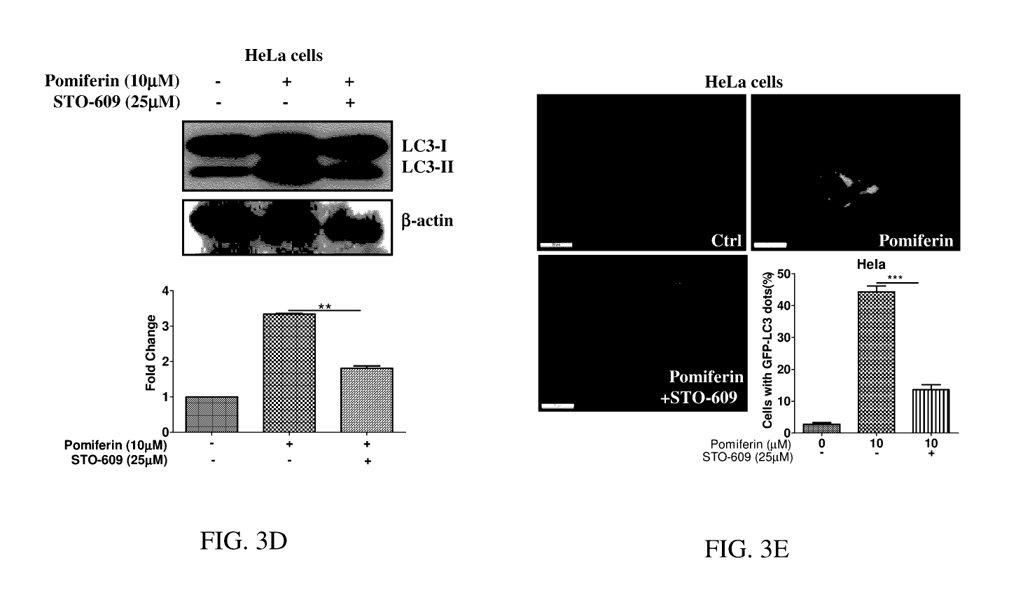

As shown in FIG. 3A, pomiferin is found to activate the phosphorylation of AMPK in a time dependent manner and this activation is also accompanied by a concomitant reduction in its mTOR downstream p70S6K phosphorylation. In order to demonstrate whether the upstream of AMPK signaling is involved in pomiferin-induced autophagy, specific inhibitors such as AMPK inhibitor, compound C and CaMKK-β inhibitor, STO-609 are used in the study. Results showed that there is a significant reduction in pomiferin-induced LC3-II conversion and GFP-LC3 puncta formation in HeLa cells treated with the presence of AMPK inhibitor (compound C) (FIGS. 3B&C) and CaMKK-β inhibitor, STO-609 (FIGS. 3D&E). These findings further suggested that pomiferin induces autophagy via activation of AMPK-mTOR signaling pathways.

### Example 5

### Pomiferin Mobilizes Cytosolic Calcium for Induction of Autophagy in HeLa Cancer Cells

Calcium Detection by Flow Cytometry Analysis.

Changes in intracellular free calcium are measured by a fluorescent dye, Fluo-3, as described previously²¹. Briefly, HeLa cells are washed twice with MEM medium after pomiferin treatment (10 μM) for various times (10 min, 0.5 h, 1 h, 2 h, 4 h). Then cell suspensions are incubated with 5 μM Fluo-3 at 37° C. for 30 min. Then the cells are washed twice with HBSS. After re-suspended cell samples are subjected to FACS analysis, at least 10,000 events are analyzed.

Detection of Autophagic Marker Protein LC3-II Conversion:

After pomiferin treatment in the presence or absence of calcium chelator, BAPTA/AM [10M], HeLa cancer cells are harvested and lysed in RIPA buffer (Cell Signaling Technologies Inc. (Beverly, Mass.). The cell lysates are then resolved by SDS-PAGE. After electrophoresis, the proteins from SDS-PAGE are transferred to nitrocellulose membrane which is then blocked with 5% non-fat dried milk for 60 minutes. The membrane is then incubated with LC3 primary antibodies (1:1000) in TBST overnight at 4° C. After that, the membrane is further incubated with HRP-conjugated secondary antibodies for 60 minutes. Finally, protein bands are visualized by using the ECL Western Blotting Detection Reagents (Invitrogen, Paisley, Scotland, UK). Quantification of autophagy GFP-LC3 Puncta: GFP-LC3 puncta formation is quantified as previously described¹³. In brief, cells grown on coverslips in a 6-well plate are incubated with 10 μM of pomiferin in the presence or absence of calcium chelator, BAPTA/AM [10M] for 24 hours, the cells are then fixed in 4% paraformaldehyde for 20 minutes at room temperature and then rinsed with PBS. Slides are mounted with FluorSave™ mounting media (Calbiochem, San Diego, Calif.) and examined by fluorescence microscopy. The number of GFP-positive cells with GFP-LC3 puncta formation is captured and examined under the Delta Vision fluorescence microscope. To quantify for autophagy, the percentage of cells with punctate GFP-LC3 fluorescence is calculated by counting the number of the cells with punctate GFP-LC3 fluorescence in GFP-positive cells. A minimum of 150 cells from 3 randomly selected fields is scored.

Results:

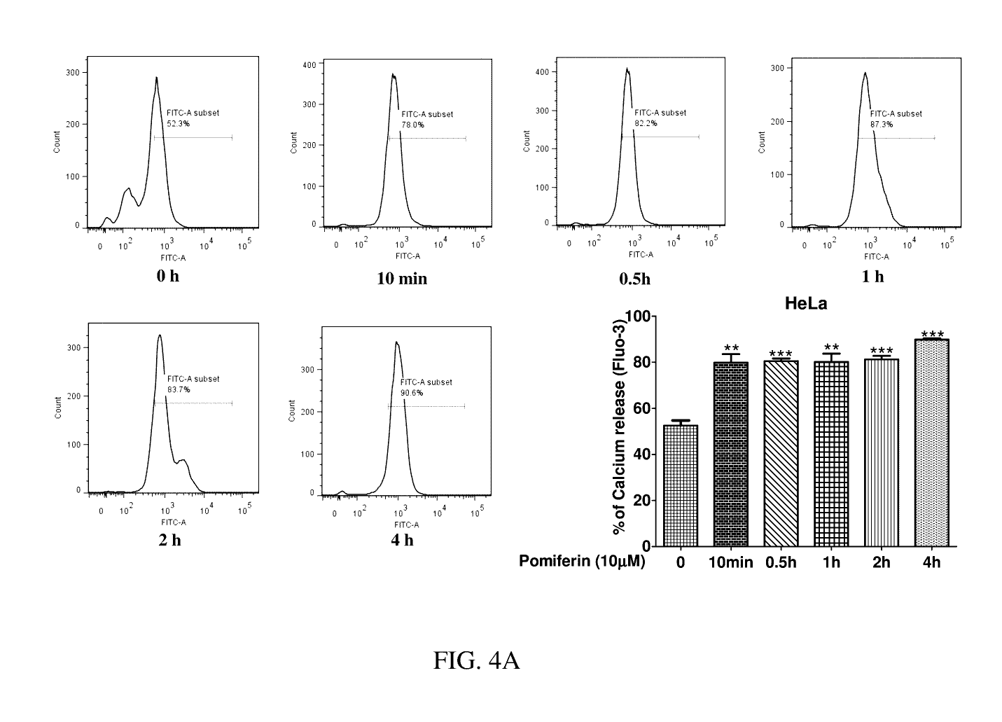

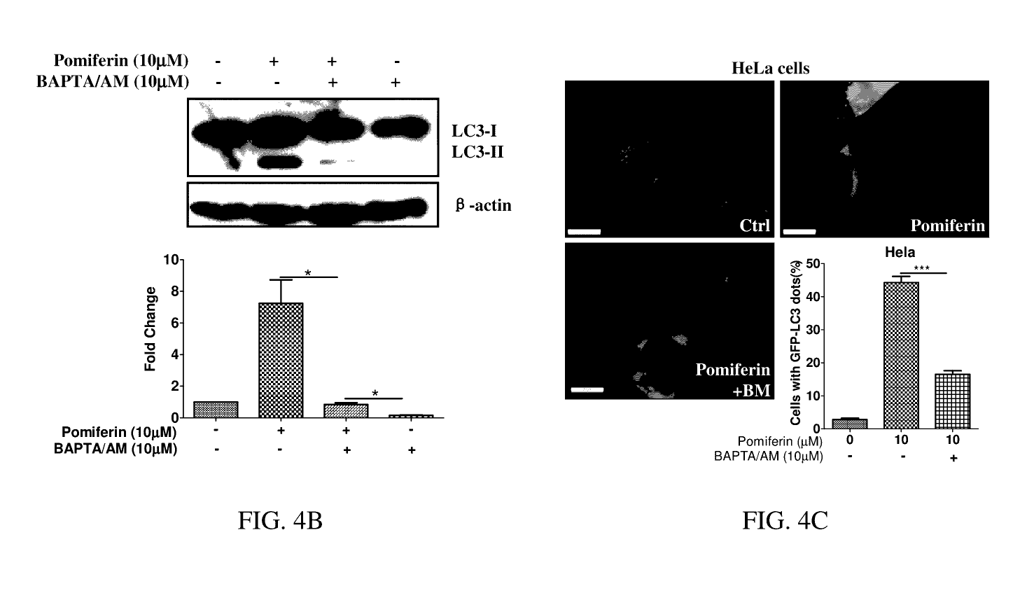

Given that calcium mobilization in cells will contribute to autophagy induction, and FIGS. 3D&E indicated that CaMKK-β is involved in pomiferin-mediated autophagy induction, suggesting that calcium may involve for pomiferin-mediated cellular functions. This example further demonstrated that pomiferin is able to increase the cytosolic calcium level in a time dependent manner as shown in FIG. 4A. To examine whether the release of cytosolic calcium would contribute autophagy, a calcium chelator, BAPTA/AM, is adopted to validate the pomiferin-induced autophagic effect. As expected, addition of BAPTA/AM could markedly suppress the pomiferin-mediated LC3-II conversion (FIG. 4B). FIG. 4C also indicated that BAPTA/AM suppressed pomiferin-mediated LC3-II conversion as compared to the same which was treated with pomiferin only. Concomitantly, the pomiferin-induced autophagic puncta formation was also inhibited in the presence of BAPTA/AM. Collectively, pomiferin induces autophagy via the mobilization of cytosolic calcium in cancer cells.

### Example 6

### Pomiferin Targets on SERCA for Cytosolic Calcium Release

Molecular Computational Docking:

The 3D structure of pomiferin is obtained from the PubChem (http://pubchem.ncbi.nlm.nih.gov). Then, the compound is preprocessed by the LigPrep²² which uses OPLS-2005 force field²³ to obtain the corresponding low energy 3D conformers. The ionized state is assigned by using Epik3 at a target pH value of 7.0±2.0. The 3D crystal structure of the sarco(endo)plasmic reticulum Ca2+ ATPase (SERCA) for molecular docking is retrieved from the Protein Data Bank (PDB ID code 2AGV)²⁴. The Protein Preparation Wizard is used to remove crystallographic water molecules, add hydrogen atoms, and assign partial charges based on OPLS-2005 force field²⁵. Energy minimization is also performed and terminated when the root-mean-square deviation (RMSD) reached a maximum value of 0.3 A. Pomiferin is docked into the thapsigargin (TG) binding site of the SERCA using Glide program²⁶ with the extra precision (XP) scoring mode. The docking grid box is defined by centering on TG in the SERCA.

Measurement of SERCA Activity:

Purified Ca²⁺ ATPase (SERCA1A) is prepared from female rabbit hind leg muscle²⁷. ATPase activity is determined using the enzyme-coupled method utilizing pyruvate kinase and lactate dehydrogenase as previously described in Michelangeli et al. (1990). All SERCA inhibition data are fitted to the allosteric dose versus effect equation using Fig P (Biosoft):

Activity=minimum activity+(maximum activity−minimum activity)/(1+([I]/IC₅₀)P).

Results:

In order to explore the possible binding poses of pomiferin in SERCA, molecular docking method was applied. The performance of molecular docking is usually evaluated by re-docking the crystal structure pose. Herein, TG in the X-ray co-crystallized complex 2AGV²⁴ is re-docked into the binding sites and the XP docking score was −7.23 kcal/mol. The RMSD of the atomic positions between the ligand and the docked pose was 1.78 Å, which means the atoms of the TG in the docked pose is coincided with the ligand atoms in the crystal structure.

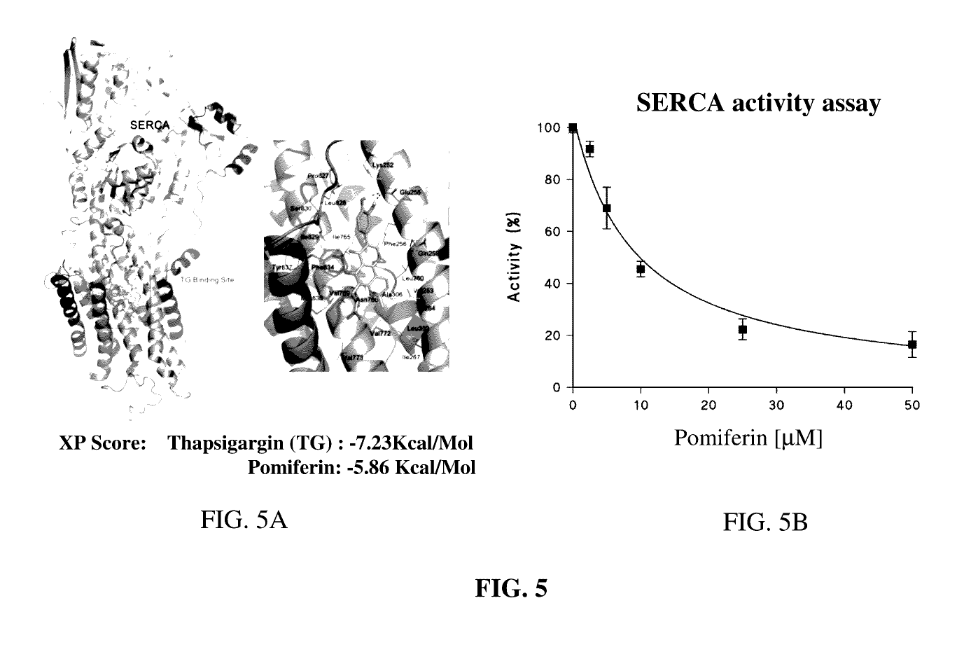

The calculated interaction energy (XP docking score) for pomiferin was −5.86 kcal/mol. FIG. 5 illustrated the structure of pomiferin docked into the SERCA TG binding site. In the predicted binding pose of the pomiferin (FIG. 5A), the hydrophobic groups of pomiferin made favorable hydrophobic effects and van der Waals interactions with residues Phe256, Leu260, Val263, Ile267, Ile264, Leu302, Val772, Val773, Ile765, Val769, Pro827, Leu828, Ile829, Ser830, and Phe834. Additionally, pomiferin was found to form hydrogen bond with residue Glu255. To ascertain whether the SERCA pump is suppressed by pomiferin, the SERCA inhibitory effect was quantified using purified rabbit skeletal muscle sarcoplasmic reticulum (SR) membranes, which measured the activity from the SERCA1A isoform in the SR membrances²⁸. Most of existing SERCA inhibitors show similar inhibitory effect in SERCA isoform^{[15,16]}. The SERCA1A pump (from rabbit skeletal muscle SR) is inhibited by pomiferin in a dose-dependent manner (FIG. 5B), which is fitted to an allosteric dose versus effect equation. Taken together, pomiferin targets on SERCA for calcium mobilization in cancer cells.

### Example 7

### Pomiferin Requires Autophagy-Related Gene 7 (Atg7) for Autophagy Induction and Induces Autophagic Cell Death

Quantification of Endogenous Autophagic LC3 Puncta in Atg7 Wild Type and Deficient MEFs:

Endogenous LC3 puncta formation is quantified as previously described². In brief, both Atg7 wild-type and deficient mouse embryonic fibroblasts (MEFs) grown on coverslips in a 6-well plate are treated with indicated concentrations of pomiferin. Both Atg7 wild-type and deficient mouse embryonic fibroblasts are then fixed in 4% paraformaldehyde for 20 minutes at room temperature and then rinsed with PBS. After washing with PBS, the cells are then incubated with anti-LC3 (1:200) in TBST (100 mM Tris HCl, pH 7.5, 150 mM NaCl, 0.05% Tween 20 and 5% BSA) overnight at 4. After washing with PBS, the cells are incubated with anti-mouse secondary antibody (TRITC) 1:200 in TBST containing 5% BSA at 37 for 1 hrs in the dark. The coverslips are then mounted with FluorSave™ mounting media (Calbiochem, San Diego, Calif., USA) for fluorescence imaging and localization of LC3 autophagosomes are captured under the API Delta Vision Live-cell Imaging System (Applied Precision Inc., GE Healthcare Company, Washington, USA). To quantify for autophagy, the percentage of cells with punctate TRITC-LC3 fluorescence is calculated by counting the number of the cells with punctate TRITC-LC3 fluorescence in TRITC-positive cells. A minimum of 150 cells from 3 randomly selected fields is scored.

Cell Culture and Flow Cytometry Analysis.

Cell viability is measured using an annexin V staining kit (BD Biosciences, San Jose, Calif., USA). Briefly, Atg7 wild-type (Atg7+/+ or Atg7-wt) and Atg7 deficient (Atg7−/− or Atg7-ko) mouse embryonic fibroblasts (MEFs) are treated with the 2 μM pomiferin for 24 h. Cells are then harvested and analysed by multiparametric flow cytometry using FITC-Annexin V and Propidium iodide staining (BD Biosciences, San Jose, Calif., USA) according to the manufacturer's instructions. Flow cytometry is then carried out using a FACSCalibur flow cytometer (BD Biosciences, San Jose, Calif., USA). Data acquisition and analysis is performed with CellQuest (BD Biosciences, San Jose, Calif., USA). Data are obtained from three independent experiments.

Results:

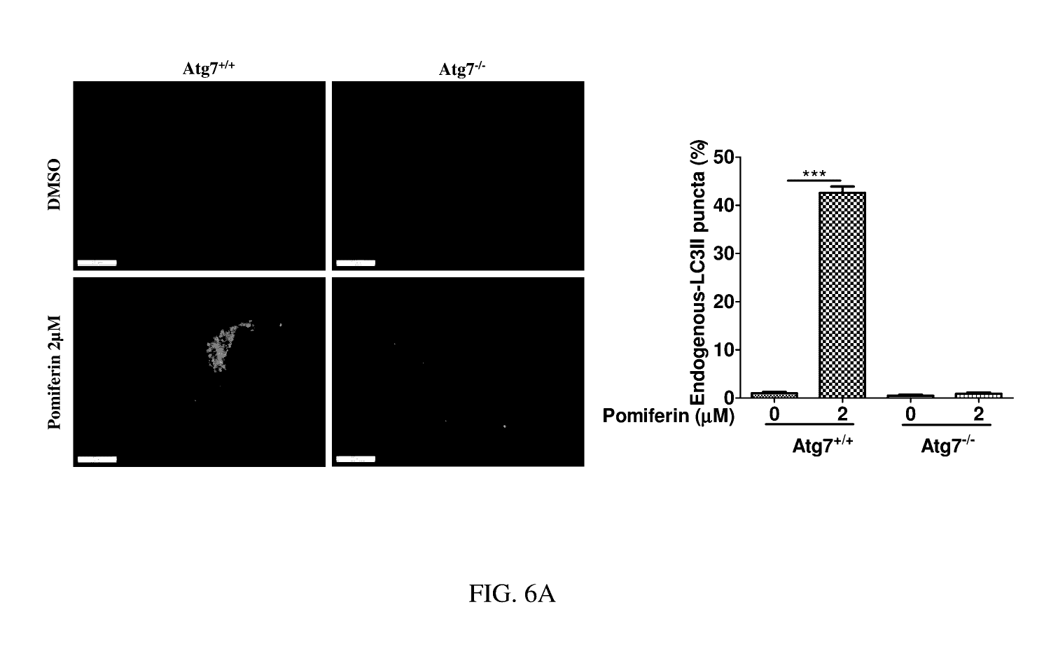

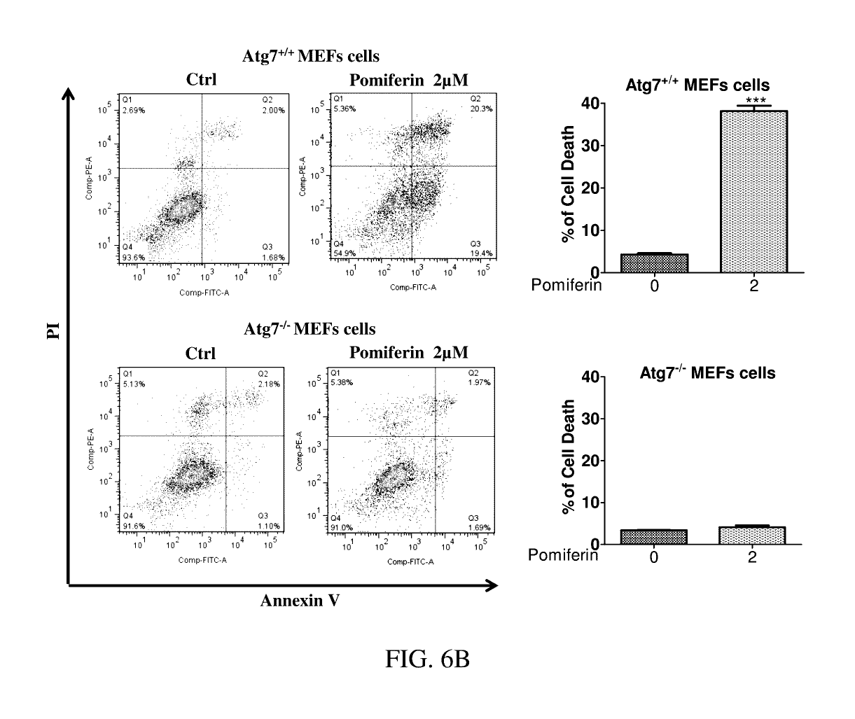

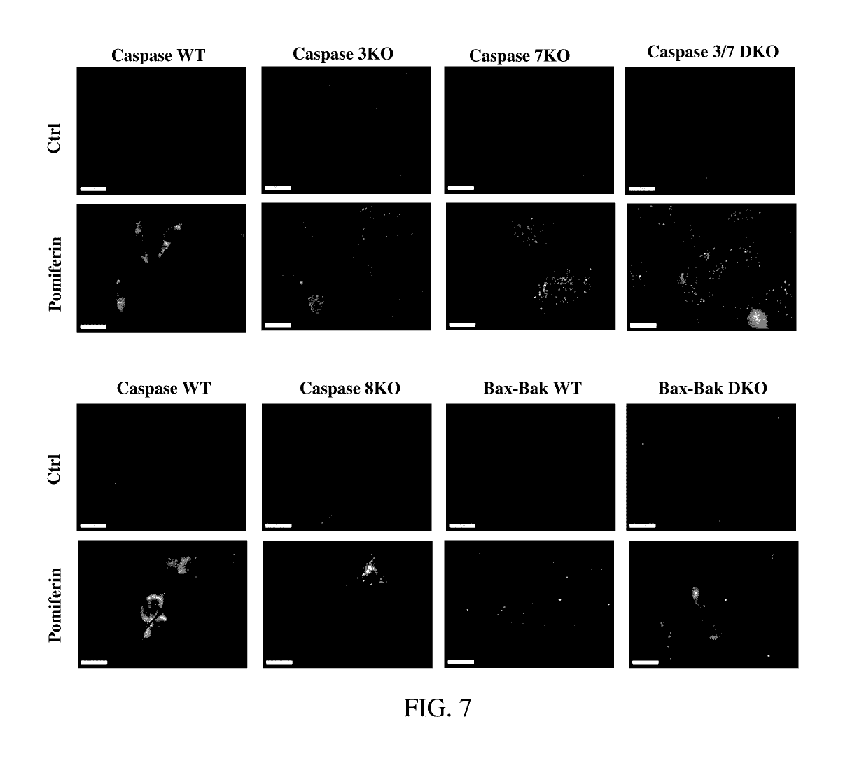

Pomiferin is found to induce TRITC-LC3 puncta formation in wild type Atg7 cells (Atg7+/+) but not in Atg7-knockout (Atg7−/−) mouse embryonic fibroblasts as shown in FIG. 6A. Thus, pomiferin works as a novel autophagy enhancer which depends on autophagy related gene, Atg7, for the induction of autophagy. To determine the role of pomiferin-induced autophagy in cell death, we adopted the Atg7 wild-type and deficient MEFs to investigate the pomiferin-mediated autophagic effect. As shown in FIG. 6B, pomiferin was found to markedly induce cell death in Atg7+/+ cells, but not in autophagy deficient cells (Atg7−/−). These findings suggest that pomiferin-mediated cell death is autophagy dependent; in other words, pomiferin is able to induce autophagic cell death.

### Example 8

### Pomiferin Exhibits Potent Cytotoxic Effect and Induces Autophagy in a Panel of Apoptosis-Resistant Cells

Cell Culture and Cytotoxicity Assay:

The test compound of pomiferin is dissolved in DMSO at a final concentration of 100 mmol/L and stored at −20° C. Cytotoxicity is assessed using the 3-(4,5-dimethylthiazol-2-yl)-2,5-diphenyltetrazolium bromide assay as previously described²⁹. 2500 of caspase wild-type (caspase WT), caspase-3 deficient (caspase 3KO), caspase-7 deficient (caspase 7KO), caspase-3/-7 deficient (caspase 3/7 DKO), caspase-8 deficient (caspase 8KO), Bax-Bak wild-type (Bak-Bak WT) and Bax-Bak double knock out (Bak-Bak DKO) mouse embryonic fibroblasts (MEFs) are seeded on 96-well plates per well. After overnight pre-incubation, the cells are exposed to different concentrations of pomiferin (namely 100, 50, 25, 12.5, 6.25, 3.125, 1.5625, 0.78, 0.39, 0.195, 0.079, 0.039 μmol/L) for 3 days. Subsequently, 10 μL of MTT reagents is added to each well and incubated at 37° C. for 4 hours, followed by the addition of 100 μL solubilization buffer (10% SDS in 0.01 mol/L HCl) and overnight incubation. Absorbance at 585 nm is determined from each well on the following day. The percentage of cell viability is calculated using the following formula: Cell viability (%)=Cells number_{treated}/Cells number_{DMSO control}×100. Data is obtained from three independent experiments.

Detection of Endogenous Autophagic LC3 Puncta in Apoptosis-Resistant MEFs:

Endogenous LC3 puncta formation is quantified as previously described². In brief, caspase wild-type (caspase WT), caspase-3 deficient (caspase 3KO), caspase-7 deficient (caspase 7KO), caspase-3/-7 deficient (caspase 3/7 DKO), caspase-8 deficient (caspase 8KO), Bax-Bak wild-type (Bak-Bak WT) and Bax-Bak double knock out (Bak-Bak DKO) MEFs are grown on coverslips in a 6-well plate are treated with 5 M of pomiferin. Both wild-type and deficient MEFs are then fixed in 4% paraformaldehyde for 20 minutes at room temperature and then rinsed with PBS. After washing with PBS, the cells are then incubated with anti-LC3 (1:200) in TBST (100 mM Tris HCl, pH 7.5, 150 mM NaCl, 0.05% Tween 20 and 5% BSA) overnight at 4. After washing with PBS, the cells are incubated with anti-mouse secondary antibody (TRITC) 1:200 in TBST containing 5% BSA at 37 for 1 hrs in the dark. The coverslips are then mounted with FluorSave™ mounting media (Calbiochem, San Diego, Calif., USA) for fluorescence imaging and localization of LC3 autophagosomes are captured under the API Delta Vision Live-cell Imaging System (Applied Precision Inc., GE Healthcare Company, Washington, USA).

Results:

Pomiferin was found to exhibit similar cytotoxic effect on both wild-type and apoptosis-resistant cells, i.e. caspase-3/-7/-8 as compared to the caspase wild-type MEFs as shown in Table 2. In addition, it also showed similar cytotoxicity in Bax-Bak DKO apoptosis-resistant cells as compared to Bax-Bak wild-type MEFs (Table 2), indicating that pomiferin is able to induce cell death in apoptosis-resistant cells. In addition, pomiferin was able to induce autophagy in all these wild-type and apoptosis-resistant cells (FIG. 7).

TABLE 2
Cell cytotoxicity of pomiferin toward a panel of
apoptosis-resistant cells in terms of IC₅₀ value:

| Cell line | Means of IC₅₀ [μM] |
|---|---|
| Caspase WT | 7.63 |
| Caspase 3KO | 2.13 |
| Caspase 7KO | 3.95 |
| Caspase 3/7 DKO | 2.21 |
| Caspase 8KO | 4.98 |
| Bax-Bak WT | 0.44 |
| Bax-Bak DKO | 0.986 |

### Example 9

### Pomiferin Shows Collateral Sensitivity in Bax-Bak DKO Apoptosis-Resistant Cells

Cell Culture and Flow Cytometry Analysis.

Cell death is measured using an annexin V staining kit (BD Biosciences, San Jose, Calif., USA). Briefly, Bax-Bak wild-type and Bax-Bak DKO MEFs are treated with the 5 μM pomiferin for 24 h. Cells are then harvested and analysed by multiparametric flow cytometry using FITC-Annexin V and Propidium iodide staining (BD Biosciences, San Jose, Calif., USA) according to the manufacturer's instructions. Flow cytometry is then carried out using a FACSCalibur flow cytometer (BD Biosciences, San Jose, Calif., USA). Data acquisition and analysis is performed with CellQuest (BD Biosciences, San Jose, Calif., USA). Data are obtained from three independent experiments.

Results:

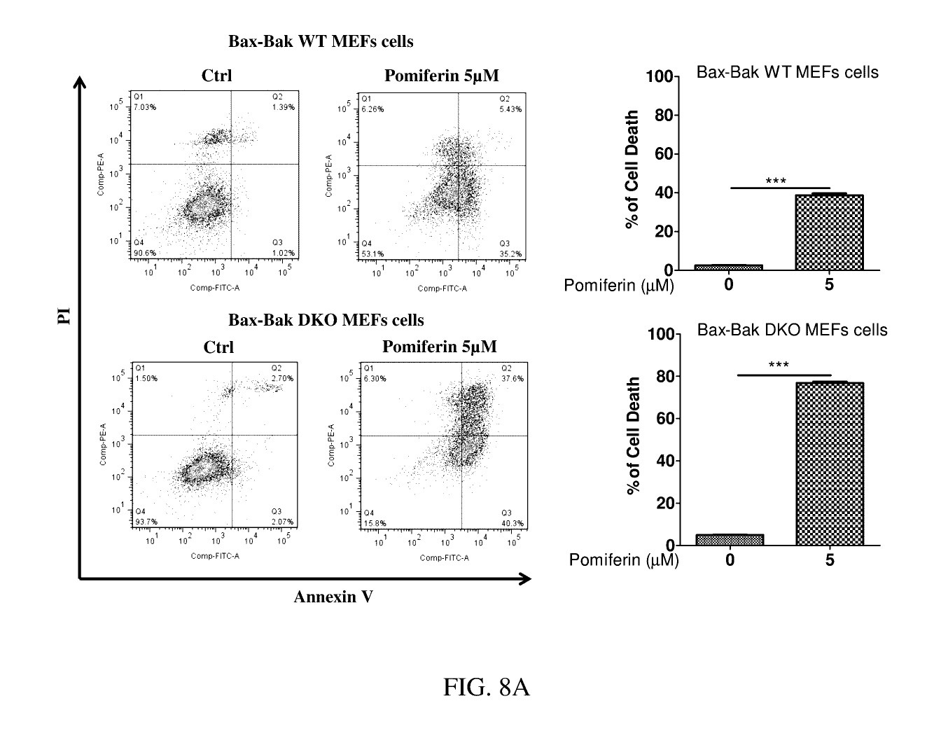

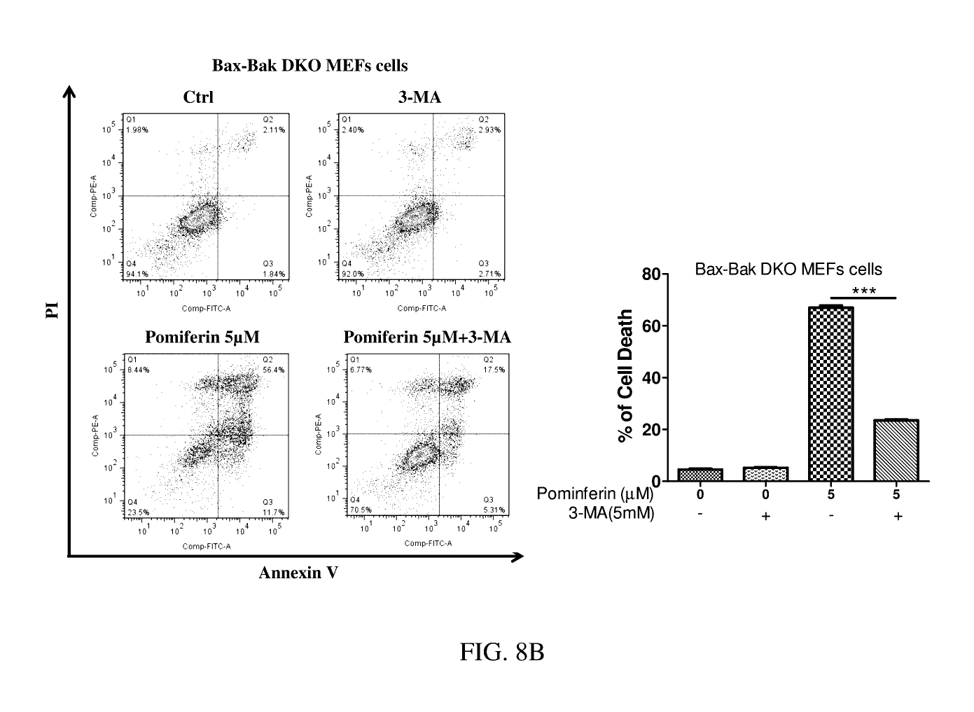

As shown in FIG. 8A, 5 μM of pomiferin only showed ˜40% of cell death in Bax-Bak wild-type MEFs, however, it demonstrated around ˜80% of cell death in Bax-Bak DKO MEFs. These findings suggested that pomiferin shows collateral sensitivity toward the apoptosis-resistant cells.

### Example 10

### Autophagic Inhibitor 3-MA Abolishes Pomiferin-Mediated Autophagic Cell Death in Apoptosis-Resistant Cells

Cell Culture and Flow Cytometry Analysis.

Cell death is measured using an annexin V staining kit (BD Biosciences, San Jose, Calif., USA). Briefly, Bax-Bak DKO MEFs are treated with the 5 μM pomiferin in the presence or absence of 5 mM autophagic inhibitor 3-MA for 24 h. Cells are then harvested and analysed by multiparametric flow cytometry using FITC-Annexin V and Propidium iodide staining (BD Biosciences, San Jose, Calif., USA) according to the manufacturer's instructions. Flow cytometry is then carried out using a FACSCalibur flow cytometer (BD Biosciences, San Jose, Calif., USA). Data acquisition and analysis is performed with CellQuest (BD Biosciences, San Jose, Calif., USA). Data are obtained from three independent experiments.

Results:

As shown in FIG. 8B, pomiferin alone significantly induced cell death in Bax-Bak DKO apoptosis-resistant cells, whereas addition of autophagic inhibitor 3-MA markedly suppressed the pomiferin-mediated cytotoxicity, suggesting that pomiferin induce cell death in apoptosis-resistant cells via autophagy induction.

### Example 11

### Pomiferin Induces Cell Death in Drug-Resistant HCT-116 p⁵³ Deficient Isogenic Colon Cancer Cells Via Calcium Mobilization and Autophagy Induction

Cell Culture and Cytotoxicity Assay:

The test compound of pomiferin is dissolved in DMSO at a final concentration of 100 mmol/L and stored at −20° C. Cytotoxicity is assessed using the 3-(4,5-dimethylthiazol-2-yl)-2,5-diphenyltetrazolium bromide assay as previously described²⁹. 4000 of drug-resistant HCT-116 p53+/+, HCT-116 p53−/− and HCT-116 p53−/− isogenic colon cancer cells are seeded on 96-well plates per well. After overnight pre-incubation, the cells are exposed to different concentrations of pomiferin (namely 100, 50, 25, 12.5, 6.25, 3.125, 1.5625, 0.78, 0.39, 0.195, 0.079, 0.039 mol/L) for 3 days. Subsequently, 10 μL of MTT reagents is added to each well and incubated at 37° C. for 4 hours, followed by the addition of 100 μL solubilization buffer (10% SDS in 0.01 mol/L HCl) and overnight incubation. Absorbance at 585 nm is determined from each well on the following day. The percentage of cell viability is calculated using the following formula: Cell viability (%)=Cells number_{treated}/Cells number_{DMSO control}×100. Data is obtained from three independent experiments.

Cell Culture and Flow Cytometry Analysis.

Cell death is measured using an annexin V staining kit (BD Biosciences, San Jose, Calif., USA). Briefly, drug-resistant HCT-116 p53−/− deficient colon cancer cells are treated with the 10 μM pomiferin in the presence or absence of 5 mM autophagic inhibitor 3-MA or 10 M calcium chelator BAPTA/AM for 24 h. Cells are then harvested and analysed by multiparametric flow cytometry using FITC-Annexin V and Propidium iodide staining (BD Biosciences, San Jose, Calif., USA) according to the manufacturer's instructions. Flow cytometry is then carried out using a FACSCalibur flow cytometer (BD Biosciences, San Jose, Calif., USA). Data acquisition and analysis is performed with CellQuest (BD Biosciences, San Jose, Calif., USA). Data are obtained from three independent experiments.

Results:

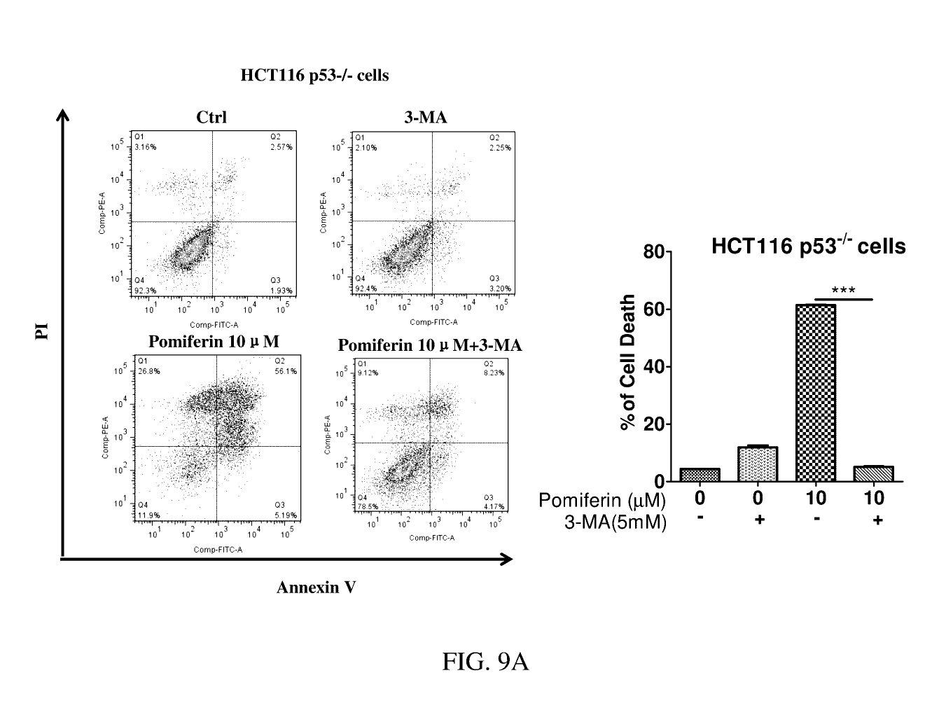

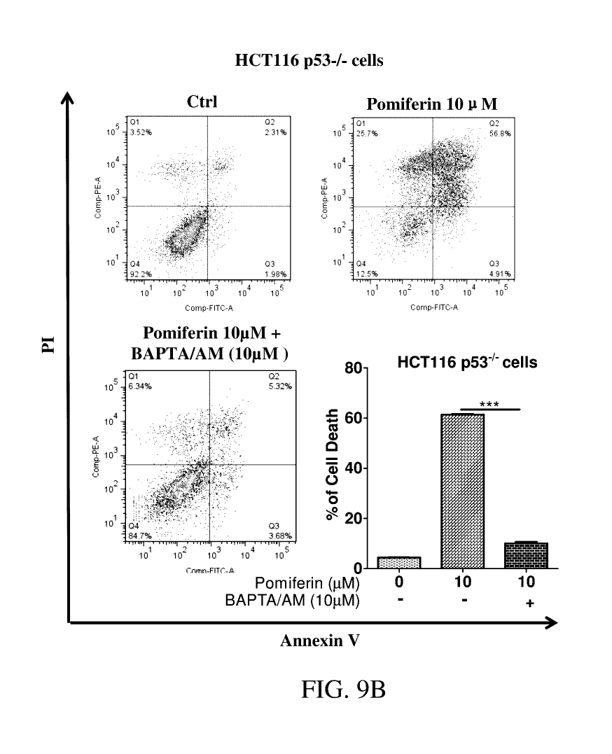

Pomiferin was found to exhibit similar cytotoxic effect on HCT-116 isogenic colon cancer cells with different p53 status (Table 3). In addition, pomiferin alone significantly induced cell death in drug-resistant HCT-116 p53−/− colon cancer cells, whereas addition of autophagic inhibitor 3-MA or calcium chelator BAPTA/AM could markedly suppress the pomiferin-induced cell death in this drug-resistant cancer (FIGS. 9A & B). Taken together, these findings suggested that pomiferin is able to kill drug-resistant cancer cells via calcium mobilization and autophagy induction.

TABLE 3
Cell cytotoxicity of pomiferin toward isogenic drug-resistant
cancer cells, HCT-116 p53, in terms of IC₅₀ value:

| Cell line | Means of IC₅₀ [μM] |
|---|---|
| HCT116 p53+/+ | 6.51 |
| HCT116 p53+/− | 5.58 |
| HCT116 p53−/− | 6.12 |

+/+wild-type HCT-116 p53;
+/−heterozygous HCT-116 p53;
−/−HCT-116 p53-deficient

### INDUSTRIAL APPLICABILITY

The present invention provides the potential use of pomiferin in developing drug for treating drug-resistant or apoptosis-resistant cancer via specific inhibition of SERCA in order to induce autophagy in the drug-resistant or apoptosis-resistant cancer cells.

### REFERENCE

The following references are incorporated herein by reference in their entirety:
1. Denmeade, S. R. & Isaacs, J. T. The SERCA pump as a therapeutic target: making a “smart bomb” for prostate cancer. Cancer Biol Ther 4, 14-22 (2005).
2. Wong, V. K., et al. Saikosaponin-d, a novel SERCA inhibitor, induces autophagic cell death in apoptosis-defective cells. Cell Death Dis 4, e720 (2013).
3. Das, S. G., et al. Ethyl 2-amino-6-(3,5-dimethoxyphenyl)-4-(2-ethoxy-2-oxoethyl)-4H-chromene-3-carboxylate (CXL017): a novel scaffold that resensitizes multidrug resistant leukemia cells to chemotherapy. ACS Chem Biol 8, 327-335 (2012).
4. Tian, F., Schrodl, K., Kiefl, R., Huber, R. M. & Bergner, A. The hedgehog pathway inhibitor GDC-0449 alters intracellular Ca2+ homeostasis and inhibits cell growth in cisplatin-resistant lung cancer cells. Anticancer Res 32, 89-94 (2012).
5. Janssen, K., et al. Inhibition of the ER Ca2+ pump forces multidrug-resistant cells deficient in Bak and Bax into necrosis. J Cell Sci 122, 4481-4491 (2009).
6. Levine, B. & Kroemer, G. Autophagy in the pathogenesis of disease. Cell 132, 27-42 (2008).
7. Pallauf, K. & Rimbach, G. Autophagy, polyphenols and healthy ageing. Ageing Res Rev 12, 237-252 (2012).
8. Sarkar, S., et al. Small molecules enhance autophagy and reduce toxicity in Huntington's disease models. Nat Chem Biol 3, 331-338 (2007).
9. Mathew, R., et al. Autophagy suppresses tumor progression by limiting chromosomal instability. Genes Dev 21, 1367-1381 (2007).
10. Liang, X. H., et al. Induction of autophagy and inhibition of tumorigenesis by beclin 1. Nature 402, 672-676 (1999).
11. Kondo, Y., Kanzawa, T., Sawaya, R. & Kondo, S. The role of autophagy in cancer development and response to therapy. Nat Rev Cancer 5, 726-734 (2005).
12. Hoyer-Hansen, M., Bastholm, L., Mathiasen, I. S., Elling, F. & Jaattela, M. Vitamin D analog EB1089 triggers dramatic lysosomal changes and Beclin 1-mediated autophagic cell death. Cell Death Differ 12, 1297-1309 (2005).
13. Law, B. Y., et al. Alisol B, a novel inhibitor of the sarcoplasmic/endoplasmic reticulum Ca(2+) ATPase pump, induces autophagy, endoplasmic reticulum stress, and apoptosis. Mol Cancer Ther 9, 718-730 (2010).
14. Gruber, J. V., Holtz, R., Sikkink, S. K. & Tobin, D. J. In vitro and ex vivo examination of topical Pomiferin treatments. Fitoterapia 94, 164-171.
15. Vesela, D., Kubinova, R., Muselik, J., Zemlicka, M. & Suchy, V. Antioxidative and EROD activities of osajin and pomiferin. Fitoterapia 75, 209-211 (2004).
16. Svasti, J., et al. Proteomic profiling of cholangiocarcinoma cell line treated with pomiferin from Derris malaccensis. Proteomics 5, 4504-4509 (2005).
17. Son, I. H., Chung, I. M., Lee, S. I., Yang, H. D. & Moon, H. I. Pomiferin, histone deacetylase inhibitor isolated from the fruits of Maclura pomifera. Bioorg Med Chem Lett 17, 4753-4755 (2007).
18. Yang, R., Hanwell, H., Zhang, J., Tsao, R. & Meckling, K. A. Antiproliferative activity of pomiferin in normal (MCF-10A) and transformed (MCF-7) breast epithelial cells. J Agric Food Chem 59, 13328-13336 (2011).
19. Bartosikova, L., et al. [Examination of the antioxidative and antidiabetic effect of pomiferin in alloxan-induced diabetes mellitus in an experiment (a pilot study)]. Ceska Slov Farm 56, 135-140 (2007).
20. Klionsky, D. J., et al. Guidelines for the use and interpretation of assays for monitoring autophagy. Autophagy 8, 445-544.
21. Liu, M. J., Wang, Z., Ju, Y., Wong, R. N. & Wu, Q. Y. Diosgenin induces cell cycle arrest and apoptosis in human leukemia K562 cells with the disruption of Ca2+ homeostasis. Cancer Chemother Pharmacol 55, 79-90 (2005).
22. Schrodinger, L. New York, N.Y. LigPrep, version 2.3. (2009).
23. Kaminski, G. A. F., R. A.; Tirado-Rives, J.; Jorgensen, W. L. Evaluation and reparametrization of the OPLS-AA force field for proteins via comparison with accurate quantum chemical calculations on peptides. J. Phys. Chem. B 105, 6474-6487 (2001).
24. Obara, K., et al. Structural role of countertransport revealed in Ca(2+) pump crystal structure in the absence of Ca(2+). Proc Natl Acad Sci USA 102, 14489-14496 (2005).
25. Epik. version 2.0, Schrodinger, LLC, New York, N.Y., (2009).
26. Glide. version 5.5, Schrodinger, LLC, New York, N.Y. (2009).
27. Michelangeli, F. & Munkonge, F. M. Methods of reconstitution of the purified sarcoplasmic reticulum (Ca(2+)-Mg2+)-ATPase using bile salt detergents to form membranes of defined lipid to protein ratios or sealed vesicles. Anal Biochem 194, 231-236 (1991).
28. Wu, K. D., Lee, W. S., Wey, J., Bungard, D. & Lytton, J. Localization and quantification of endoplasmic reticulum Ca(2+)-ATPase isoform transcripts. Am J Physiol 269, C775-784 (1995).
29. Wong, V. K., Zhou, H., Cheung, S. S., Li, T. & Liu, L. Mechanistic study of saikosaponin-d (Ssd) on suppression of murine T lymphocyte activation. J Cell Biochem 107, 303-315 (2009).

What is claimed is:

1. A composition for treating cancers comprising an effective amount of a compound of formula (I): 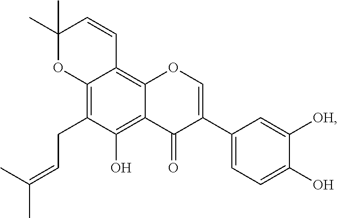 [SMILES: CC(C)=CCc1c2c(c3occ(-c4ccc(O)c(O)c4)c(=O)c3c1O)C=CC(C)(C)O2]
a pharmaceutically acceptable carrier, salt, buffer, water, or a combination thereof, said effective amount of the compound ranges from 0.44 to 20.3 μM.

2. The composition of claim 1, wherein said effective amount of the compound of formula (I) ranges from 3.82 to 20.3 μM and said cancers comprise cervical cancer, breast cancer, liver cancer, lung cancer, and prostate cancer, or other cancer cells thereof.

3. The composition of claim 1, wherein said effective amount of the compound of formula (I) ranges from 0.44 to 7.63 μM and said cancers comprise apoptosis-resistant cells or cancer cells thereof.

4. The composition of claim 1, wherein said effective amount of the compound of formula (I) ranges from 5.58 to 6.51 μM and said cancers comprise drug-resistant cells or cancer cells thereof.

5-12. (canceled)

13. A method of inducing cell death in isogenic drug-resistant colon cancer cells comprising contacting a compound of formula (I): 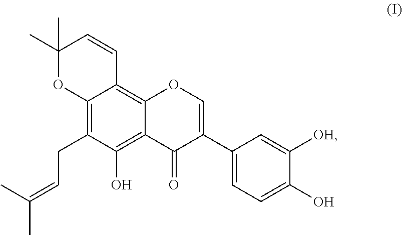 [SMILES: CC(C)=CCc1c2c(c3occ(-c4ccc(O)c(O)c4)c(=O)c3c1O)C=CC(C)(C)O2]
with said cancer cells in a concentration from 5.58 to 6.51 μM.

14. The method of claim 13, wherein said contacting mobilizes cytosolic calcium release.

15. The method of claim 14, wherein said cytosolic calcium release is via inhibition of sarcoplasmic endoplasmic reticulum calcium ATPase (SERCA) activity in said cancer cells.

16. (canceled)

17. (canceled)

18. The method of claim 13, wherein said isogenic drug-resistant colon cancer cells comprise drug resistant HCT-116 p53 deficient isogenic colon cancer cells.

19. The method of claim 13, wherein said cell death comprises apoptosis and autophagic cell death.

20. The method of claim 19, wherein said apoptosis and autophagic cell death induced by said compound is dependent on activation AMPK-mTOR signaling cascade.

21. A method of inducing cell death and/or autophagy in apoptosis-resistant cancer cells from human or mouse origin comprising contacting a compound of formula (I): 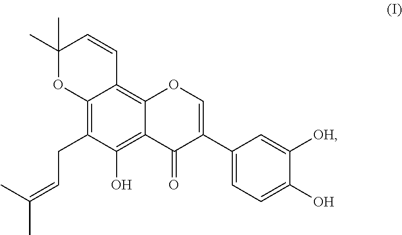 [SMILES: CC(C)=CCc1c2c(c3occ(-c4ccc(O)c(O)c4)c(=O)c3c1O)C=CC(C)(C)O2]
with said cancer cells in a concentration from 0.44 to 7.63 μM.

22. The method of claim 21, wherein said contacting mobilizes cytosolic calcium release.

23. The method of claim 22, wherein said cytosolic calcium release is via inhibition of sarcoplasmic endoplasmic reticulum calcium ATPase (SERCA) activity in said cancer cells.

24. The method of claim 21, wherein said apoptosis-resistant cancer cells comprise caspase wild-type (caspase WT), caspase-3 deficient (caspase 3KO), caspase-7 deficient (caspase 7KO), caspase-3/-7 deficient (caspase 3/7 DKO), caspase-8 deficient (caspase 8KO), Bax-Bak wild-type (Bax-Bak WT) and Bax-Bak double knock out (Bax-Bak DKO) mouse embryonic fibroblasts (MEFs).

25. The method of claim 24, wherein said apoptosis-resistant cancer cells are Bax-Bak DKO MEFs and the concentration of said compound is about 5 μM.

26. The method of claim 21, wherein said cell death comprises apoptosis and autophagic cell death.

27. The method of claim 26, wherein said apoptosis and autophagic cell death induced by said compound is dependent on activation AMPK-mTOR signaling cascade.
